(19) United States
(12) Patent Application Publication
(10) Pub. No.: US 2003/0109546 A1
(43) Pub. Date: Jun. 12, 2003

(54) Treatment of psychotic disorders comprising co-therapy with anticonvulsant derivatives and atypical antipsychotics

(76) Inventor: Wayne S. Fenton, Rockville, MD (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/131,277

(22) Filed: Apr. 23, 2002

Related U.S. Application Data
(60) Provisional application No. 60/286,765, filed on Apr. 26, 2001.
(60) Provisional application No. 60/301,661, filed on Jun. 28, 2001.

(51) Int. Cl. A61K 31/47; A01N 43/42

(52) U.S. Cl. 514/310

(57) ABSTRACT

Treatment of psychotic disorders such as schizophrenia, schizophreniform and schizoaffective disorders comprising co-therapy with an anticonvulsant derivative and a typical antipsychotic.

### CROSS REFERENCE TO RELATED APPLICATION

This application claims priority from U.S. provisional application Serial No. 60/286,765, filed Apr. 26, 2001 and U.S. provisional application Serial No. 60/301,661, filed Jun. 28, 2001, the contents of which are hereby incorporated by reference.

### BACKGROUND OF THE INVENTION

Compounds of Formula (I):

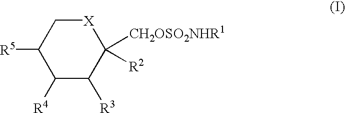

are structurally novel antiepileptic compounds that are highly effective anticonvulsants in animal tests (MARYANOFF, B. E, NORTEY, S. O., GARDOCKI, J. F., SHANK, R. P. AND DODGSON, S. P. J. Med. Chem. 1987, 30, 880-887; MARYANOFF, B. E., COSTANZO, M. J., SHANK, R. P., SCHUPSKY, J. J., ORTEGON, M. E., AND VAUGHT J. L. Bioorg. Med. Chem. Lett. 1993, 3, 2653-2656; SHANK, R. P., GARDOCKI, J. F., VAUGHT, J. L., DAVIS, C. B., SCHUPSKY, J. J., RAFFA, R. B., DODGSON, S. J., NORTEY, S. O., MARYANOFF, B. E. Epilepsia 1994, 35, 450-460; MARYANOFF B E, COSTANZO M J, NORTEY S O, GRECO M N, SHANK R P, SCHUPSKY J J, ORTEGON M P, VAUGHT J L. J. Med. Chem. 1998, 41, 1315-1343). These compounds are covered by three U.S. patents: U.S. Pat. Nos. 4,513,006, 5,242,942, and 5,384,327. One of these compounds 2,3:4,5-bis-O-(1-methylethylidene)-β-D-fructopyranose sulfamate known as topiramate has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world.

Compounds of Formula (I) were initially found to possess anticonvuisant activity in the traditional maximal electroshock seizure (MES) test in mice (SHANK, R. P., GARDOCKI, J. F., VAUGHT, J. L., DAVIS, C. B., SCHUPSKY, J. J., RAFFA, R. B., DODGSON, S. J., NORTEY, S. O., and MARYANOFF, B. E., Epilepsia 1994, 35, 450-460). Subsequent studies revealed that Compounds of Formula I were also highly effective in the MES test in rats. Topiramate was also found to effectively block seizures in several rodent models of epilepsy (J. NAKAMURA, S. TAMURA, T. KANDA, A. ISHII, K. ISHIHARA, T. SERIKAWA, J. YAMADA, and M. SASA, Eur. J. Pharmacol. 1994, 254, 83-89), and in an animal model of kindled epilepsy (A. WAUQUIER and S. ZHOU, Epilepsy Res. 1996, 24, 73-77).

More recently, Shank, R P in U.S. Pat. No. 5,753,693 discloses the use of compounds of formula (I) for the treatment of bipolar disorder. van Kammen, D P in WIPO publication WO 00/32183 discloses the use of compounds of formula (I) for the treatment of schizophrenia.

Psychotic disorders are those that are predominantly characterized by psychosis. Psychotic disorders include schizophrenia, schizophreniform disorder, schizoaffective disorder, delusional disorder, brief psychotic disorder, shared psychotic disorder, psychotic disorder as a result of a general medical condition, substance-induced psychotic disorder, and psychotic disorder not otherwise specified (Diagnostic and Statistical Manual of Mental Disorders, Ed. 4^{th}, American Psychiatric Association, Washington, D.C. 1994; Kaplan & Sadock's Comprehensive Textbook of Psychiatry, Seventh Edition, Volume I, Lippincott Williams & Wilkins: Philadelphia, pp. 825, 2000).

According to the American Psychiatric Association, the term “psychotic” means grossly impaired in reality testing. Gross impairment in reality testing is defined as existing when individuals incorrectly evaluate the accuracy of their perceptions and thoughts, and make incorrect inferences about external reality, even in the face of contrary evidence. The term “psychotic” is also appropriate when behavior is so disorganized that it is reasonable to infer that reality testing is grossly disturbed, for example, when there is markedly incoherent speech without apparent awareness by the person that the speech is not understandable, or when agitated, inattentive, and disoriented behavior is observed in the phencyclidine psychotic disorder (Diagnostic and Statistical Manual of Mental Disorders, Ed. 4^{th}, American Psychiatric Association, Washington, D.C. 1994; Kaplan & Sadock's Comprehensive Textbook of Psychiatry, Seventh Edition, Volume I, Lippincott Williams & Wilkins: Philadelphia, pp. 825, 2000).

Schizophrenia is a group of illnesses that are phenomenologically and etiologically heterogeneous and which are characterized by perturbations of language, perception, thinking, social activity, affect, and volition, but not pathognomonic features. The syndrome commonly begins in late adolescence, has an insidious onset, and, classically, a poor outcome, progressing from social withdrawal and perceptual distortions to a state of chronic delusions and hallucinations. Patients with schizophrenia may present with positive symptoms, which include but are not limited to, conceptual disorganization, disorganized speech, delusions, hallucinations and/or with negative symptoms which include, but are not limited to, loss of function, anhedonia, paucity of speech, decreased emotional expression, impaired concentration, and diminished social engagement. “Negative” symptoms predominate in one-third of the schizophrenic population and are associated with a poor long-term outcome and a poor response to drug treatment. However, marked variability in the course and individual character of symptoms is typical. Moreover, suicidal behavior is a serious problem among patients with schizophrenia (Harrison's Online www.harrisons.com dated Oct. 12, 2000; Chapter 385; Diagnostic and Statistical Manual of Mental Disorders, Ed. 4^{th}, American Psychiatric Association, Washington, D.C. 1994; Kaplan & Sadock's Comprehensive Textbook of Psychiatry, Seventh Edition, Volume I, Lippincott Williams & Wilkins: Philadelphia, pp. 825, 2000).

Schizophrenia can also be defined by the specific symptomatology present, although such distinctions do not generally correlate with either course of illness or response to treatment. Additionally, many individuals have symptoms of more than one type. The four main symptom subtypes are catatonic, paranoid, disorganized, and residual. Catatonic-type patients present with profound changes in motor activity, negativism, and echolalia or echopraxia. Paranoid-type patients present with a prominent preoccupation with a specific delusional system. Disorganized-type is associated with disorganized speech and behavior and may be accompanied by a superficial or silly affect. Residual-type is characterized by negative symptomatology and includes the absence of delusions, hallucinations, or motor disturbance.

A diagnosis of schizophreniform disorder is reserved for patients who meet the symptom requirements of schizophrenia, but not the duration requirements. Schizoaffective disorder is an illness with co-existing, but independent, schizophrenic (psychotic) and mood components.

With a diagnosis of schizophrenia, schizophreniform or schizoaffective disorder, prognosis generally depends on the response to antipsychotic medication. Antipsychotic agents remain the first line therapy for both acute and maintenance treatment of schizophrenia and are generally effective in the treatment of hallucinations, delusions, and thought disorders, regardless of etiology. Antipsychotic treatments include:

(a) typical or traditional antipsychotics, such as phenothiazines (eg, chlorpromazine, thioridazine, fluphenazine, perphenazine, trifluoperazine, levomepromazin), thioxanthenes (eg, thiothixene, flupentixol), butyrophenones (eg, haloperidol), dibenzoxazepines (eg, loxapine), dihydroindolones (eg, molindone), substituted benzamides (eg, sulpride, amisulpride), and the like; and

(b) a typical antipsychotics, such as clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, nemonapride, SR-31742 (Sanofi), CX-516 (Cortex), SC-111 (Scotia), NE-100 (Taisho), and the like.

Conventional antipsychotic agents are effective in approximately 70 percent of patients presenting with a first episode. Improvement may be observed within hours or days, but full remission usually requires 6 to 8 weeks. The choice of antipsychotic is largely based on the side-effect profile or on a past personal or family history of a favorable response to the drug in question.

### DISCLOSURE OF THE INVENTION

It has now been found that compounds of the following formula (I):

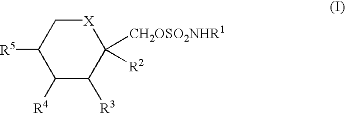

wherein X is O or CH₂, and R¹, R², R³, R⁴ and R⁵ are as defined hereinafter administered in co-therapy with a typical antipsychotics are useful in treating psychotic disorders.

In an embodiment of the invention, one or more compound(s) of formula (I) administered in co-therapy with one or more a typical antipsychotic(s) are useful in treating schizophrenia, schizophreniform disorder and/or schizoaffective disorder. In a further embodiment of the invention, one or more compound(s) of formula (I) administered in co-therapy with one or more a typical antipsychotics are useful in treating schizophrenia.

### DETAILED DESCRIPTION OF THE PREFERRED EMBODIMENTS

As used herein, the term “psychotic disorder” shall include schizophrenia, schizophreniform disorder, schizoaffective disorder, delusional disorder, brief psychotic disorder, shared psychotic disorder, psychotic disorder due to a general medical condition, substance-induced psychotic disorder, and psychotic disorder not otherwise specified schizophrenia. Preferably, the term “psychotic disorders” shall include schizophrenia, schizophreniform disorder, delusional disorder, brief psychotic disorder, shared psychotic disorder, psychotic disorder due to a general medical condition, substance-induced psychotic disorder, and psychotic disorder not otherwise specified schizophrenia. More preferably the “psychotic disorder” is schizophrenia.

As used herein, the term “schizophrenia” shall include schizophrenia, catatonic-type schizophrenia, paranoid-type schizophrenia, disorganized type schizophrenia and residual-type schizophrenia.

As used herein the term “schizophreniform disorder” shall mean a disorder with symptoms consistent with schizophrenia, but whose duration is not consistent with schizophrenia.

As used herein the term “schizoaffective disorder” shall mean a disorder with co-existing, but independent, schizophrenic (psychotic) and mood components.

Treatment options for psychotic disorders including schizophrenia, schizophreniform disorder, schizoaffective disorder and others, include the administration of typical or traditional antipsychotics and/or a typical antipsychotic or combinations thereof. Atypical antipsychotics are characterized by less extrapyramidal symptoms, especially dystonias, associated with therapy as compared to a typical antipsychotic such as haloperidol. Prototypical a typical antipsychotics also differ from the typical antipsychotics with the following characteristics: (a) greater efficacy in the treatment of overall psychopathology in patients with schizophrenia nonresponsive to typical antipsychotics; (b) greater efficacy in the treatment of negative symptoms of schizophrenia; and (c) less frequent and quantitatively smaller increases in serum prolactin concentrations associated with therapy (Beasley, et al., Neuropsychopharmacology, 14(2), 111-123, (1996)).

Atypical antipsychotics include, but are not limited to:

2-methyl-4-(4-methyl-1-piperazinyl)-10H-thieno[2,3-b]benzodiazepine, known as olanzapine and described in U.S. Pat. No. 5,229,382 as useful for the treatment of schizophrenia, schizophreniform disorder, acute mania, mild anxiety states and psychosis; with a recommended dosage of 5-30 mg/day, preferably 5-10 mg/day (Physician's Desk Reference; Kaplan & Sadock's Comprehensive Textbook of Psychiatry, Seventh Edition, Volume II, Lippincott Williams & Wilkins: Philadelphia, 2000);

8-chloro-11-(4-methyl-1-piperazinyl)-5H-dibenzo[b,e][1,4]diazepine, known as clozapine and disclosed in U.S. Pat. No. 3,539,573, with clinical efficacy in the treatment of schizophrenia described in Hanes, et al., Psychopharmacological Bulletin, 24, 62 (1988)); with a recommended dosage of 12.5-600 mg/day, preferably 250-450 mg/day (Physician's Desk Reference; Kaplan & Sadock's Comprehensive Textbook of Psychiatry, Seventh Edition, Volume II, Lippincott Williams & Wilkins: Philadelphia, 2000);

3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, known as risperidone and described in U.S. Pat. No. 4,804,663 as useful for the treatment of psychotic diseases; with a recommended dosage of 0.25-16 mg/day, preferably 1-16 mg/day, more preferably 2-8 mg/day (Physician's Desk Reference; Kaplan & Sadock's Comprehensive Textbook of Psychiatry, Seventh Edition, Volume II, Lippincott Williams & Wilkins: Philadelphia, 2000);

1-[2-[3-[5-chloro-1-(4-fluorophenyl)-1H-indol-3-yl]-1-piperidinyl]ethyl]imidazolidin-2-one, known as sertindole and disclosed in U.S. Pat. No. 4,710,500, with U.S. Pat. No. 5,112,838 and U.S. Pat. No. 5,238,945 disclosing the use of sertindole for the treatment of schizophrenia; with a starting dose of 4 mg/day, with increases of 4 mg every other day up to 24 mg/day, with final recommended dosage range of 12 to 20 mg/day (Kaplan & Sadock's Comprehensive Textbook of Psychiatry, Seventh Edition, Volume II, Lippincott Williams & Wilkins: Philadelphia, pp. 2467-2468, 2000);

5-[2-(4-dibenzo[b,f][1,4]thiazepin-11-yl-1-piperazinyl)ethoxy]ethanol, known as quetiapine and disclosed in U.S. Pat. No. 4,879,288 for the treatment of schizophrenia; with a recommended dosage of 25-800 mg/day, preferably 150-750 mg/day (Physician's Desk Reference; Kaplan & Sadock's Comprehensive Textbook of Psychiatry, Seventh Edition, Volume II, Lippincott Williams & Wilkins: Philadelphia, 2000);

5-[2-[4-(1,2-dibenzoisothiazol-3-yl)-1-piperazinyl]ethyl]-6-chloro-1,3-dihydro-2H-indol-2-one, known as ziprasidone and disclosed in U.S. Pat. No. 4,831,031 and U.S. Pat. No. 5,312,925, with its utility in the treatment of schizophrenia disclosed in U.S. Pat. No. 4,831,031; with a recommended dosage of 40-160 mg/day, with a preferred dosage for maintenance treatment and prevention of relapse of 40 to 60 mg twice a day (Kaplan & Sadock's Comprehensive Textbook of Psychiatry, Seventh Edition, Volume II, Lippincott Williams & Wilkins: Philadelphia, pp. 2470-2471, 2000).

As used herein, the term “subject”, refers to an animal, preferably a mammal, most preferably a human, who is the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. More particularly, in the present invention directed to co-therapy comprising administration of one or more compound(s) of formula (I) and one or more a typical antipsychotic(s), “therapeutically effective amount” shall mean that amount of the combination of agents taken together so that the combined effect elicits the desired biological or medicinal response. For example, the therapeutically effective amount of co-therapy comprising administration of a compound of formula (I) and an a typical antipsychotic would be the amount of the compound of formula (I) and the amount of the a typical antipsychotic that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the a typical antipsychotic individually may or may not be therapeutically effective.

As used herein, the term “co-therapy” shall mean treatment of a subject in need thereof by administering one or more compounds of formula (I) with one or more a typical antipsychotics, wherein the compound(s) of formula (I) and the a typical antipsychotic(s) are administered by any suitable means, simultaneously, sequentially, separately or in a single pharmaceutical formulation. Where the compound(s) of formula (I) and the a typical antipsychotic(s) are administered in separate dosage forms, the number of dosages administered per day for each compound may be the same or different. The compound(s) of formula (I) and the a typical antipsychotic(s) may be administered via the same or different routes of administration. The compound(s) of formula (I) and the a typical antipsychotic(s) may be administered via the same or different routes of administration. Suitable examples of methods of administration are orally, intravenous (iv), intramuscular (im), and subcutaneous (sc). Compounds may also be administrated directly to the nervous system including, but not limited to the intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) and the a typical antipsychotic(s) may be administered according to simultaneous or alternating regimens, at the same or different times during the course of the therapy, concurrently in divided or single forms.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

As used herein, the term “composition” is intended to encompass a product comprising the specified ingredients in the specified amounts, as well as any product which results, directly or indirectly, from combinations of the specified ingredients in the specified amounts.

The sulfamates of the invention are of the following formula (I):

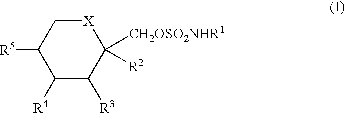

wherein

X is CH₂ or oxygen;

R¹ is hydrogen or alkyl; and

R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II):

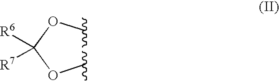

wherein

R⁶ and R⁷ are the same or different and are hydrogen, lower alkyl or are alkyl and are joined to form a cyclopentyl or cyclohexyl ring.

R₁ in particular is hydrogen or alkyl of about 1 to 4 carbons, such as methyl, ethyl and iso-propyl. Alkyl throughout this specification includes straight and branched chain alkyl. Alkyl groups for R², R³, R⁴, R⁵, R⁶ and R⁷ are of about 1 to 3 carbons and include methyl, ethyl, iso-propyl and n-propyl. When X is CH₂, R⁴ and R⁵ may combine to form a benzene ring fused to the 6-membered X-containing ring, i.e., R⁴ and R⁵ are defined by the alkatrienyl group ═C—CH═CH—CH═.

A particular group of compounds of formula (I) is that wherein X is oxygen and both R² and R³ and R⁴ and R⁵ together are methylenedioxy groups of the formula (II), wherein R⁶ and R⁷ are both hydrogen both alkyl or combine to form a spiro cyclopentyl or cyclohexyl ring, in particular where R⁶ and R⁷ are both alkyl such as methyl. A second group of compounds is that wherein X is CH₂ and R⁴ and R⁵ are joined to form a benzene ring. A third group of compounds of formula (I) is that wherein both R² and R³ are hydrogen.

The compounds of formula (I) may be synthesized by the following methods:

(a) Reaction of an alcohol of the formula RCH₂OH with a chlorosulfamate of the formula ClSO₂NH₂ or ClSO₂NHR¹ in the presence of a base such as potassium t-butoxide or sodium hydride at a temperature of about −200 to 25° C. and in a solvent such as toluene, THF, or dimethylformamide wherein R is a moiety of the following formula (III):

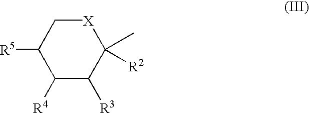

(b) Reaction of an alcohol of the formula RCH₂OH with sulfurylchloride of the formula SO₂Cl₂ in the presence of a base such as triethylamine or pyridine at a temperature of about −40° to 25° C. in a solvent such as diethyl ether or methylene chloride to produce a chlorosulfate of the formula RCH₂OSO₂Cl.

The chlorosulfate of the formula RCH₂OSO₂Cl may then be reacted with an amine of the formula R¹NH² at a temperature of abut 40° to 25° C. in a solvent such as methylene chloride or acetonitrile to produce a compound of formula (I). The reaction conditions for (b) are also described by T. Tsuchiya et al. in Tetrahedron Lett., 1978, 3365.

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by M. Hedayatullah in Tetrahedron Lett. 1975, 2455. The azidosulfate is then reduced to a compound of formula (I) wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula (II) may be obtained by the method of R. F. Brady in Carbohydr. Res. 1970, 14, 35 or by reaction of the trimethylsilyl enol ether of a R⁶COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by G. L. Larson et al. in J. Org. Chem. 1973, 38, 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such a diglyme, THF or toluene at a temperature of about 0° to 100° C., e.g. as described by H. O. House in “Modern Synthetic Reactions”, 2nd Ed., pages 45 to 144 (1972).

The compounds of formula I: may also be made by the process disclosed U.S. patents: U.S. Pat. Nos. 4,513,006, 5,242,942, and 5,384,327, which are incorporated by reference herein.

The compounds of formula I include the various individual isomers as well as the racemates thereof, e.g., the various alpha and beta attachments, i.e., below and above the plane of the drawing, of R², R³, R⁴ and R⁵ on the 6-membered ring. Preferably, the oxygen of the methylenedioxy group (II) are attached on the same side of the 6-membered ring.

The ability of the compounds of formula I administered as co-therapy with a typical antipsychotics to treat psychotic disorders is based on recent case studies.

### EXAMPLE 1

The patient was a 26 year old single Caucasian male, diagnosed with schizoaffective disorder, who presented with a variety of problems including disorganized behavior, suspiciousness, ideas of reference, stereotyped and obsessional thinking, lack of motivation, poor hygiene, poverty of speech, oppositional behavior and intermittent suicide attempts. The patient spent virtually the entire day in bed and was awake through the night.

Between the ages of 9 and 26, the patient had multiple hospitalizations and was treated unsuccessfully with a variety of medications and combinations including Mellaril (thioridazine) Tegretol (carbamazepine), Klonopin (clonazepam), Zoloft (sertraline), Depakote (divalproex sodium), Trilafon (perphenazine), Paxil (paroxetine hydrochloride), Wellbutrin (bupropion hydrochloride), clozapine, risperidone, fluvoxamine, Ativan (lorazepam), Eskalith (lithium) and olanzapine. Despite continuous psychiatric and pharmacological treatment, the patient remained substantially disabled by withdrawal, apathy, poverty of speech, poor hygiene, anhedonia and suspiciousness.

At the time of initial examination, the patient was on a regimen of fluvoxamine (a serotonin selective reuptake inhibitor) at 200 mg, Depakote at 1000 mg, lorazepam (a benzodiazepine) at 2 mg and olanzapine at 10 mg daily. In addition, the patient was seen weekly in individual psychotherapy. There were no additional active medical problems.

The patient was started on topiramate with dosages increased over a period of six weeks to 400 mg/day. Over a period of 8 weeks, a significant improvement in negative symptoms, including eye contact, attention, concentration, poverty of speech, grooming, hygiene and withdrawal, was noted. In addition, the patient's residual positive symptoms, including primary suspiciousness and ideas of reference, were improved and the patient became less fearful of interacting with other. Patient's social functioning was improved, with the patient able to hold part-time employment.

After 5 months of treatment, topiramate dosage was decreased to 300 mg/day with improvements maintained. After 10 months of treatment, improvement was still sustained, with the patient taking 300 mg topiramate, 7.5 mg olanzapine, 400 mg fluvoxamine and 1 mg lorazepam daily. Affect, personal hygiene, motivation and ability to communicate remained improved and positive symptoms remained under control. The patient maintained his part-time employment and engaged in productive social activity several times a week. After more than 2 years of treatment, the patient remained improved on a daily regimen of 300 mg topiramate, 7.5 mg olanzapine and 400 mg fluvoxamine. (Lorazepam was removed with sustained improvement.)

### EXAMPLE 2

### Results from Open Label Pilot Study on Topiramate as Adjunctive Therapy in the Treatment of Schizophrenia

Patient 1

The patient was a 50 year old male who met DSM-IV diagnostic criteria for chronic undifferentiated schizophrenia. He had a 31 year history of illness marked by multiple hospitalizations and was attending an outpatient partial hospitalization program 5 days a week. The patient had been maintained on a stable regimen of 20 mg olanzapine prior to initiation of the study. Despite the olanzapine treatment, the patient exhibited prominent negative symptoms including flattened affect, emotional and passive/apathetic social withdrawal, and concrete thinking. Although the patient lived in a group home with other residents, he preferred to spend most of his free time alone, watching television or staying in his room, and went to bed between 7 and 8 o'clock each night. The patient had limited social interactions in the partial hospitalization program, initiating contact with only a few friends. The patient was intrusive during groups and was unable to empathize with others within the group. The patient also demonstrated marked stereotyped and monothematic thinking, talking about golf inappropriately in response to general questioning on frequent occasions. In addition, the patient demonstrated mild positive symptoms such as suspiciousness, grandiosity, and guilt, but these minimally affected his thinking and behavior. The patient exhibited noticeably disorganized speech, irrelevant, tangential and circumstantial responses were elicited during clinical and semi-structured interviews.

The patient was begun on topiramate at an initial dose of 25 mg/day, titrating to an optimal dosage level of 175 mg/day, which was given in two divided doses during an eight week maintenance medication phase of a clinical trial. During treatment, topiramate was well tolerated; the patient denied the emergence of side-effects, and vital signs and laboratory values remained within the normal range.

While on the optimal dosage of topiramate, the patient showed a decrease in the Negative Scale score of 7 points from baseline. The patient did not show any consistent, significant change in the Positive or General Scales of the Positive and Negative Syndrome Scale (PANSS). While on topiramate, the patient reported feeling more alert and energetic, “brighter, more chipper and had a lot more thoughts”. He became more active in social situations at his group home, gained a greater awareness of others and his impact on their relationship with him and stayed up later, until about 11 o'clock each night. He also became less stereotyped in his speech, with very infrequent references to his previous recurrent theme of golf. After 12 weeks of adjunctive therapy with topiramate, the patient also appeared more realistic about his golfing skills, stating than he was an “average” golfer, as opposed to his previous assertions about winning several club tournaments.

Upon completion of the study protocol, the patient's topiramate was discontinued, resulting in an increase in the Negative Scale score. At a follow-up, the patient exhibited more flattened affect, more emotional and passive/apathetic social withdrawal and more stereotyped thinking. Moreover, the patient reported that upon discontinuation of topiramate treatment, he had a return of what he termed his “thorazine shuffle” with slowed gait, aching legs, feelings of sedation and stated that his mind was “closed off”. The patient also resumed going to bed around 6:30 or 7:00 pm each night.

Patients 2 & 3

Two additional patients also completed the study, but did not respond significantly to adjunctive topiramate therapy with regards to negative symptoms.

One patient demonstrated a limited decrease in negative symptoms that fluctuated during the drug maintenance period, although the patient's Negative Scale score never exceeded his baseline measurement.

The second patient demonstrated a significant decrease in negative symptoms, however his topiramate dosage was lowered from 100 mg/day to 75 mg/day and the patient was dropped from the study. The decreased topiramate dosage was necessary because of the emergence of difficulties with verbal fluency and word finding, a side effect potentially attributable to topiramate.

However, both patients displayed decreases in positive symptoms over the course of the trial.

### EXAMPLE 3

The patient (Mr. R) was a 52-year old morbidly obese, Caucasian male, who was first hospitalized in 1966 for psychotic symptoms, leading to a psychiatric diagnosis of schizophrenia. His primary symptoms consisted of a set of strange and elaborate ideas about transmitting his thoughts to others, thoughts that he had invented “invisible steel” which was being used by the CIA and aliens, and a high degree of suspiciousness about the discriminatory intent of his clergyman and the local police. He denied any overt hallucinations, although he acknowledged “weird noises” associated with aliens attempting to contact him. At initial hospitalization, the patient was also profoundly confused and disorganized in his thought processes. Over the next two decades he was hospitalized four more times with essentially this same clinical picture, eventually being admitted in 1989 to a state hospital for long-term treatment where he remains to this day.

During his five hospitalizations the patient was treated with a variety of anti-psychotic medications, often in various combinations and dosages. In 1994 he was characterized as being treatment refractory according to the Kane criteria and enrolled in an NIMH sponsored study of three different doses of clozapine. As a participant in this study the patient received 100, 300, 600 mg, each for a 3-month period. Later he received 900 mg of clozapine, which led to a seizure. On the basis of regular symptom ratings for the NIMH study, and in the opinion of the treatment team, the patient did not show any meaningful response to clozapine treatment.

The patient was nonetheless continued on a 100 mg dose of clozapine, with the intent of using a variety of different augmentation strategies to possibly improve his clinical state. The newer a typical medications, risperidone and then olanzapine were added at standard doses to the clozapine treatment, with little effect other than the emergence of severe tremors with risperidone. In 1999 Depakote (divalproex sodium) was added (up to the level of 5000 mg) to his clozapine treatment, with little benefit although he was continued for some time on this combination. In 2000, the patient participated in a study of “clozapine augmentation with low dose risperidone”, with the same lack of treatment benefit and tremors. Throughout this period, the patient's strange and bizarre ideas persisted. He remained emotionally withdrawn, and required regular PRN medication to reduce his agitation on the ward.

Toward the end of the year 2000, the patient was treated with a drug regimen of 100 mg of clozapine, 2 mg of risperidone, and 750 mg of valproic acid. At this time, the patient agreed to augment his treatment with topiramate. The valproic acid was gradually withdrawn, and on Dec. 5, 2000 the patient was started on 50 mg of topiramate. The topiramate was increased at a rate of 50 mg per week up to a daily dosage of 200 mg where he remained for over four months. Within the first 2-3 weeks of topiramate treatment several important clinical changes were observed. First, the patient's level of preoccupation with his delusional thoughts was obviously reduced. Rather than being very certain about his ability to transmit thought to others he now is not sure that this is possible. He was also no longer certain that he actually invented “invisible steel”, but instead mostly only believed that he once thought it, although occasionally the delusional belief breaks though into his thinking. His level of suspiciousness was also decreased. Second, as a result of treatment with topiramate the patient's agitation was decreased and PRN medication for agitation was no longer required. Third, the patient was able to engage in a logical conversation, with only mild evidence of tangential thinking.

In short, the period that included topiramate treatment was the only period during the patient's decade long hospitalization that any significant progress was made in improving his clinical condition. In the opinion of the treatment team, it was the addition of topiramate that led to a clear and important degree of improvement in the patient's clinical state.

### EXAMPLE 4

The patient (Mr. C) was a 45-year old, Caucasian male, who was first hospitalized in 1983 on an involuntary basis-due to his inability to care for himself. Upon admission, he was described as paranoid, suspicious, agitated, and delusional, and was given the diagnosis of schizophrenia, undifferentiated type. He expressed persecutory delusions saying that the Mafia was going to hurt him. He heard voices telling him that someone was going to rape his mother. After one year of hospitalization, he was discharged into the community, where he soon became unstable resulting in two suicide attempts by hanging and two more short-term hospitalizations. In 1988 the patient was admitted into a state hospital for long-term treatment where he remains to this day. Over the past decade or more, his clinical picture had remained unchanged. He had been treated with numerous anti-psychotic medications, including the newer a typical medicines with little or no benefit. His high level of agitation and persecutory delusions often propelled him into confrontations with staff and other patients, and he was regularly treated with PRN medications or placed into seclusion for his behavior. At the end of the year 2000, he was being treated with haloperidol at 100 mg/mL every four weeks, risperidone at 3 mg/day, olanzapine at 20 mg/day and valproic acid at 2000 mg/day.

Toward the end of 2000, the valproic acid was gradually withdrawn, although the haloperidol and risperidone were continued at the same dose, and on Dec. 21, 2000 the patient was started on an initial dose of 50 mg of topiramate. The topiramate dosage was titrated at a rate of 50 mg per week up to a daily dosage of 150 mg, at which dose the patient remained for a period of 4 months. Within the first month of treatment with topiramate several important clinical changes began to emerge. First, the patient's level of agitation reduced dramatically. Within 1-2 weeks, the patient was no longer in seclusion and the use of PRN medication for agitation became very rare. Second, the patient's expressions persecutory delusions or suspiciousness decreased, and he was able to join into group activities on the unit. Third, the patient exhibited fewer negative symptoms and was observed in conversations with other patients on the unit.

In short, the period of treatment that included topiramate was the only period during this decade long hospitalization when any significant progress had been made in improving his clinical condition. The intensity of his positive symptoms, such as auditory hallucinations and delusions of persecution were reduced in occurrence and severity and he was less socially withdrawn.

Thus, for treating a psychotic disorder a compound of formula (I) may be administered as co-therapy with one or more a typical antipsychotics. Preferably, the compound of formula (I) is administered in amount in the range of about 10 to about 650 mg/day. More preferably, the compound of formula (i) is administered in an amount in the range of about 10 to about 325 mg once or twice daily.

To prepare the pharmaceutical compositions of this invention, one or more sulfamate compounds of formula (I) are intimately admixed with a pharmaceutical carrier according to conventional pharmaceutical compounding techniques, which carrier may take a wide variety of forms depending on the form of preparation desired for administration, e.g., i.v. sterile injectable formulations will be prepared using appropriate solubilizing agents. A unit dose would contain about 15 to 200 mg of the active ingredient. Topiramate is currently available for oral administration in round tablets containing 25 mg, 100 mg or 200 mg of active agent. The tablets contain some or all of the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating a psychotic disorder in a subject in need thereof comprising co-therapy with a therapeutically effective amount of a compound of the formula I: 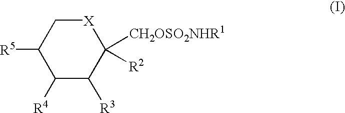
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II): 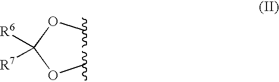
wherein
R⁶ and R⁷ are the same or different and are hydrogen, lower alkyl or are alkyl and are joined to form a cyclopentyl or cyclohexyl ring;
and one or more a typical antipsychotics.

2. The method of claim 1 wherein the psychotic disorder is selected from the group consisting of schizophrenia, schizophreniform disorder, schizoaffective disorder, delusional disorder, brief psychotic disorder, shared psychotic disorder, psychotic disorder due to a general medical condition, substance-induced psychotic disorder and psychotic disorder not otherwise specified.

3. The method of claim 1 wherein the compound of formula (I) is topiramate.

4. The method of claim 3, wherein the amount of the compound of formula (I) is from about 10 to about 650 mg.

5. The method of claim 4, wherein the amount of the compound of formula (I) is from about 10 to about 325 mg once or twice daily.

6. The method of claim 1, wherein the a typical antipsychotic is selected from the group consisting of clozapine, risperidone, olanzapine, quetiapine, zotepine, ziprasidone, iloperidone, perospirone, blonanserin, ORG-5222 (Organon), sonepiprazole, aripiprazole, nemonapride, SR-31742 (Sanofi), CX-516 (Cortex), SC-111 (Scotia), NE-100 (Taisho) and sertindole.

7. The method of claim 6, wherein the a typical antipsychotic is selected from the group consisting of olanzapine, clozapine, risperidone and quetiapine.

8. The method of claim 7, wherein the a typical antipsychotic is risperidone.

9. The method of claim 8, wherein the amount of the a typical antipsychotic is from about 0.25 to about 16 mg daily.

10. The method of claim 9, wherein the amount of the a typical antipsychotic is from about 2 to about 8 mg daily.

11. A method for treating schizophrenia in a subject in need thereof comprising co-therapy with a therapeutically effective amount of a compound of formula (I) 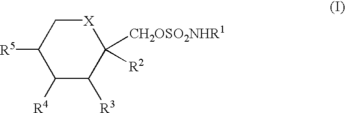
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II): 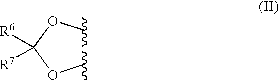
wherein
R⁶ and R⁷ are the same or different and are hydrogen, lower alkyl or are alkyl and are joined to form a cyclopentyl or cyclohexyl ring;
and one or more a typical antipsychotics.

12. The method of claim 11, wherein the compound of formula (I) is topiramate.

13. The method of claim 12, wherein the amount of the compound of formula (I) is from about 10 to about 650 mg.

14. The method of claim 12, wherein the amount of the compound of formula (I) is of from about 10 to about 325 mg once or twice daily.

15. The method of claim 11, wherein the a typical antipsychotic is selected from the group consisting of clozapine, risperidone, olanzapine, quetiapine, zotepine, ziprasidone, iloperidone, perospirone, blonanserin, ORG-5222 (Organon), sonepiprazole, aripiprazole, nemonapride, SR-31742 (Sanofi), CX-516 (Cortex), SC-111 (Scotia), NE-100 (Taisho) and sertindole.

16. The method of claim 15, wherein the a typical antipsychotic is olanzapine, clozapine, risperidone and quetiapine.

17. The method of claim 16, wherein the a typical antipsychotic is risperidone.

18. The method of claim 17, wherein the amount of the a typical antipsychotic is from about 0.25 to about 16 mg daily.

19. The method of claim 17, wherein the amount of the a typical antipsychotic is from about 2 to about 8 mg daily.
